(19) United States
(12) Patent Application Publication
(10) Pub. No.: US 2005/0026151 A1
(43) Pub. Date: Feb. 3, 2005

(54) Simultaneous generation of multiple chemiluminescent signals on solid supports

(76) Inventors: John C. Voyta, Sudbury, MA (US); Robert M. Smith, Stow, MA (US); Gary P. Schroth, San Ramon, CA (US); Alison L. Sparks, North Andover, MA (US); Brooks N. Edwards, Cambridge, MA (US)

Correspondence Address: Supervisor, Patent Prosecution Services;PIPER RUDNICK LLP, 1200 Nineteenth Street, N.W., Washington, DC, 20036-2412, US

(21) Appl. No.: 10/620,333

(22) Filed: Jul. 17, 2003

(51) Int. Cl. C12Q 1/68

(52) U.S. Cl. 435/6

(57) ABSTRACT

A chemiluminescent assay to determine the presence and/or amount of one or more labeled target molecules in a sample is described in which the surface layer of a solid support is contacted with a composition comprising first and second chemiluminescent substrates capable of being activated by first and second enzymes, respectively. A plurality of probes are disposed on the surface layer in discrete areas. At least some of the probes are bound to a first enzyme conjugate comprising the first enzyme and at least some of the probes are bound to a second enzyme conjugate comprising the second enzyme. The resulting chemiluminescent signals are then detected. The method can be used to compare two biological samples (e.g., mRNA populations from different cells) on the same support surface or to provide a chemiluminescent control signal for normalizing chemiluminescent assay data from a biological sample.

This application is related to U.S. patent application Ser. No. 10/046,730, filed Jan. 17, 2002, pending, and U.S. patent application Ser. No. 10/050,188, filed Jan. 14, 2002, pending (published as U.S. patent application Publication No. 2002/0110828 A1 on Aug. 15, 2002). This application is also related to U.S. patent application Ser. No. 10/462,742, filed on Jun. 17, 2003, pending. Each of these applications is incorporated herein by reference in its entirety.

### BACKGROUND

1. Technical Field

The subject matter of the present application relates generally to methods of conducting biological assays. More specifically, the subject matter of the present application pertains to methods of performing chemiluminescent assays on solid supports wherein two different chemiluminescent signals are simultaneously generated.

2. Background of the Technology

Microarray technology provides a useful tool for conducting biological assays. A microarray comprises a large number of different probes each of which are immobilized in different discrete areas on a substrate. For nucleic acid assays, the probes can be nucleic acid or oligonucleotide probes. When a sample is contacted with the microarray, molecules in the sample (i.e., target molecules) can hybridize to probes having complementary or substantially complementary sequences. Detection of the position of the hybridized target molecule on the array (e.g., by detecting a label on the target molecule) indicates the presence of a particular sequence in the sample. Due to the large number of different probes present in a microarray, biological assays on microarrays can be conducted in a massively parallel fashion. Microarrays have therefore proven extremely useful in screening, profiling, and sequencing nucleic acid samples.

Assays conducted on microarrays typically employ fluorescently labeled targets. Fluorescent labels can provide high spatial resolution since the signal is generated by a species (i.e., the fluorescer) which is attached to the support either directly or through a probe-target interaction and which is therefore not free to migrate during the assay. In contrast to fluorophore-labeled targets, the use of enzyme labeled targets and chemiluminescent substrates results in a signaling species (i.e., the activated substrate) which is not attached to the support and which is therefore free to migrate during the assay. Migration of the chemiluminescent species during the assay can reduce the spatial resolution of the assay and can result in inaccurate assay data. As a result, chemiluminescent detection of enzyme labeled targets on microarrays has not been widely employed.

A need still exists, however, for improved methods of detecting chemiluminescent signals from solid supports, particularly from microarrays comprising higher feature density signal generating regions in applications involving multianalyte detection.

### SUMMARY

According to a first embodiment of the invention, a method of detecting chemiluminescent emissions on a solid support is provided which includes: contacting a surface layer of the solid support with a composition comprising a first chemiluminescent substrate capable of being cleaved by a first enzyme to produce a first chemiluminescent signal and a second chemiluminescent substrate capable of being cleaved by a second enzyme to produce a second chemiluminescent signal and detecting chemiluminescent emissions from the surface layer of the solid support. A plurality of probes are disposed in a plurality of discrete areas on the surface layer at a density of at least 50 discrete areas per cm². At least some of the probes are bound to a first enzyme conjugate comprising the first enzyme and at least some of the probes are bound to a second enzyme conjugate comprising the second enzyme.

According to a second embodiment of the invention, a composition comprising a first chemiluminescent substrate capable of being cleaved by a first enzyme to produce a first chemiluminescent signal and a second chemiluminescent substrate capable of being cleaved by a second enzyme to produce a second chemiluminescent signal is also provided. According to this embodiment of the invention, the first and second chemiluminescent signals are different. The composition can be a buffered solution. The composition can also comprise a chemiluminescent enhancing material.

### DETAILED DESCRIPTION OF EMBODIMENTS

According to one embodiment of the invention, a method of detecting chemiluminescent emissions on a solid support is provided which comprises: contacting a surface layer of the solid support with a substrate composition comprising a first chemiluminescent substrate capable of being cleaved by a first enzyme to produce a first chemiluminescent signal and a second chemiluminescent substrate capable of being cleaved by a second enzyme to produce a second chemiluminescent signal. Chemiluminescent emissions from the surface layer of the solid support are then detected. A plurality of probes are disposed in a plurality of discrete areas on the surface layer. At least some of the probes are bound to a first enzyme conjugate comprising the first enzyme and at least some of the probes are bound to a second enzyme conjugate comprising the second enzyme.

The discrete areas can comprise one or more control probes. The first enzyme conjugate can be bound to a control probe and the second enzyme conjugate can be bound to a probe for a target molecule. According to this embodiment of the invention, the signal from the first chemiluminescent substrate can be used as a chemiluminescent control signal. For example, the first chemiluminescent signal can be used to quantify the amount of the bound target molecule (i.e., the amount of the second chemiluminescent signal) by, for example, comparing the intensity of the first chemiluminescent signal to the intensity of the second chemiluminescent signal.

Alternatively, the discrete areas can comprise one or more analyte probes. The first enzyme conjugate can be bound to a probe for a first target molecule and the second enzyme conjugate can be bound to a probe for a second target molecule.

A plurality of different probes can be disposed on the support surface in different discrete areas. Detection of the first and second chemiluminescent signals can comprise detecting the location on the support surface of first and second chemiluminescent signals.

Control probes can also be located in one or more discrete areas on the support surface. According to this embodiment of the invention, control probes can be co-located in one or more of the same discrete areas as probes for a target molecule.

When the target molecules in the sample are nucleic acids, the discrete areas can comprise oligonucleotide or nucleic acid probes. Alternatively, the probes can be polypeptides or other biomolecules capable of binding to target biomolecules of interest.

According to a further embodiment of the invention, the support surface can be contacted with a sample comprising first target molecules labeled with a first label and second target molecules labeled with a second label prior to contacting the support surface with the substrate composition. The first target molecules can be labeled with the first enzyme to form the first enzyme conjugate and the second target molecules are labeled with the second enzyme to form the second enzyme conjugate. Alternatively, the first target molecules can be labeled with a moiety capable of binding to the first enzyme conjugate and the second target molecules can be labeled with a moiety capable of binding to the second enzyme conjugate.

The first target molecules can comprise a first pool of target nucleic acids and the second target molecules comprise a second pool of target nucleic acids. The first and second pools of target nucleic acids can, for example, each comprise mRNA transcripts of one or more genes or nucleic acids derived from mRNA transcripts of one or more genes. In particular, the first and second pools of target nucleic acids can each comprise cDNA or cRNA derived from mRNA transcripts. The concentration of target nucleic acids in the first and second pools of target nucleic acids can be proportional to the expression level of the genes encoding the target nucleic acid.

The support surface can also comprise a fluorescent label. The fluorescent label can be imaged upon excitation (e.g., with an LED array) to localize the array elements and to provide data for the normalization of the quantitative chemiluminescence data from the array.

The first chemiluminescent signal and the second chemiluminescent signal can, according to one embodiment, have different emission maxima. Detection of the first and second chemiluminescent signals can be accomplished using filtering (e.g., optical filtering). For example, emissions from the support surface including first and second chemiluminescent signals can be filtered with a first filter adapted to reduce the intensity of the second chemiluminescent signal relative to the intensity of the first chemiluminescent signal. The first chemiluminescent signal can then be detected. The combined signal from the support surface can then be filtered with a second filter adapted to reduce the intensity of the first chemiluminescent signal relative to the intensity of the second chemiluminescent signal and the second chemiluminescent signal detected. Alternatively, the first and second chemiluminescent signals can be detected simultaneously by filtering the combined signal from the support surface with first and second filters.

According to a further embodiment of the invention, a composition comprising both a first chemiluminescent substrate capable of being cleaved by a first enzyme to produce a first chemiluminescent signal and a second chemiluminescent substrate capable of being cleaved by a second enzyme to produce a second chemiluminescent signal is also provided. The first and second chemiluminescent signals are different. The composition can be a buffered solution. The buffer can be selected to optimize simultaneous emissions from each of the chemiluminescent substrates. The composition can also include a chemiluminescent quantum yield enhancing agent, additives, and/or counterions. These components can also be chosen to optimize simultaneous emissions from each of the chemiluminescent substrates.

Detection of the chemiluminescent signals can be performed using any suitable detection technique. For example, chemiluminescence can be detected using a charge coupled device (i.e., a CCD) and a scanning system comprising a confocal microscope.

The composition comprising the first and second chemiluminescent substrates can be contacted with the surface layer in the presence of a chemiluminescent enhancing material and/or a chemiluminescent enhancing additive. The use of chemiluminescent enhancing materials and chemiluminescent enhancing additives in solid phase chemiluminescent assays is disclosed in copending U.S. patent application Ser. No. 10/462,742 (Attorney Docket No. 9550-013-27), filed on Jun. 17, 2003, which application is herein incorporated by reference in its entirety. Any of the materials and techniques disclosed in this application can be used. For example, the chemiluminescent quantum yield enhancing material and/or enhancement additive can be incorporated into the solid support prior to contacting the solid support with the substrate. Alternatively, the chemiluminescent quantum yield enhancing material and/or enhancement additive can be included in the substrate composition.

Exemplary chemiluminescent quantum yield enhancing materials which can be used are disclosed in U.S. Pat. No. 5,145,772, which is hereby incorporated by reference in its entirety. Exemplary chemiluminescent enhancement additives which can be used are disclosed in U.S. Pat. No. 5,547,836, which is also hereby incorporated by reference in its entirety.

As set forth above, some of the probes disposed on the support surface can be control probes. According to this embodiment of the invention, the sample can contain a known amount of an enzyme labeled control target and the substrate composition can contain a chemiluminescent substrate capable of being cleaved by the enzyme label on the control target (i.e., a control chemiluminescent substrate). Cleavage of the enzyme labile group on the control chemiluminescent substrate results in a chemiluminescent control signal. According to this embodiment of the invention, the amount of an analyte can be quantified by comparing the intensity of the chemiluminescent control signal to the intensity of a chemiluminescent signal derived from enzyme labeled analyte bound to the support surface. The location of the chemiluminescent control signal on the support surface can also be determined and used to locate features on the support surface.

According to an alternative embodiment of the invention, a fluorescent control signal can be used in conjunction with the multiple chemiluminescent signals. According to this embodiment of the invention, the two different chemiluminescent signals could be used to assay two different target molecules (e.g., two different pools of target nucleic acids).

As set forth above, according to an embodiment of the invention, two different chemiluminescent substrates are used. The two different chemiluminescent substrates can have well separated emission maxima. For example, the emission maxima of the two substrates when activated should be far enough apart to enable efficient filter based (e.g., optical filter based) discrimination of the signal. The two enzymes used should also be capable of specifically hydrolyzing their respective substrates and should be capable of operating in the same buffer. Also, the two enzymes used should not require additives that could interfere with or inhibit the reaction between the other enzyme and its corresponding substrate.

Each of the enzymes can be used to label target molecules in the sample. Alternatively, one of the enzyme labels can be used for a chemiluminescent control target.

According to one embodiment of the invention, one enzyme can be used to track the hybridization of labeled target molecules (e.g., cDNA or cRNA derived from cellular mRNA) to specific features on an array and the second enzyme can track the hybridization of an internal control target (ICT) to each feature on an array. The chemiluminescent signal from the internal control could then be used to locate each individual feature on the array and/or to normalize the signal from labeled analyte at each position on the array.

In another embodiment of the invention, each of the enzyme labels could be used to track the hybridization of labeled target molecules (e.g., cDNA or cRNA derived from two different sets of cellular mRNA) to specific features on an array. For example, each of the enzymes can be conjugated to a different set of nucleic acids. The labeled nucleic acids can then be allowed to hybridize to probes (e.g., oligonucleotide or nucleic acid probes) on the support surface.

The use of a chemiluminescent control rather than a fluorescent control in a chemiluminescent assay may offer certain advantages. For example, the use of fluorescence may introduce errors due to the different nature of the two systems. In particular, in the case of fluorescent detection, the emitting species is attached to the surface of the solid support. In the three-dimensional environment of a solid support, the fluorescent control can be buried in a fold, pore or cavity of the solid support and thereby be inaccessible for excitation, thus lowering the signal correlating to the fluorescent control. In the chemiluminescent system, emission may occur from the product of an enzyme reaction, where the emitting species is not attached to the solid support and thereby is accessible for activation by attached labeled probe in the three-dimensional environment of the solid support. Therefore, the chemiluminescent control can give an increased signal relative to the fluorescent control.

When a chemiluminescent assay is performed using a chemiluminescent signal (i.e., a CL/CL system), the normalization signal and the analyte signal are both chemiluminescent signals. Since the signal from each of the activated enzyme substrates (i.e., control and analyte) is from an emitting species not attached to the solid support and accessible for activation by attached labeled probe and control, the resulting CL/CL system may have better correlation between the normalization and analyte signals than an FL/CL system wherein normalization of the chemiluminescent signal is performed with a bound fluorescent control that may be obscured from excitation. For example, data that has been obtained in a chemiluminescent system using a chemiluminescent control signal for normalization can have lower coefficients of variation. This improved statistical performance can enable improved gene expression quantitation, better cross tissue comparisons and other benefits.

A fluorescent control signal, however, can also be used. According to this embodiment of the invention, the sample can contain a known amount of a fluorescent labeled control target. The amount of an analyte can be quantified by comparing the intensity of the fluorescent control signal to the intensity of a chemiluminescent signal derived from an enzyme labeled analyte bound to the support surface. The location of the fluorescent control signal on the support surface can also be determined and used to locate features on the support surface. When a fluorescent control is used, two different chemiluminescent substrates can be used to simultaneously assay two analytes each labeled with a different enzyme.

The solid support surface can comprise a plurality of different analyte probes each capable of binding with a different analyte. Groups of each of the probes can be disposed on the support surface in different discrete areas (e.g., in an array format). In this manner, the location of the signal on the surface of the solid support can be used to indicate the particular analyte being detected. In the case of nucleic acid detection, the array can comprise a plurality of different oligonucleotide or nucleic acid probes capable of hybridizing to substantially complementary nucleic acid sequences in the sample. According to this embodiment of the invention, detecting can comprise determining the location on the support surface of the chemiluminescent signals. The location of a chemiluminescent signal on the support surface can be determined using one or more enzyme labeled (e.g., chemiluminescent) or fluorescent control targets as set forth above.

If a control probe is used, the control probe can be located in one or more discrete areas on the support surface. For example, the control probe can be disposed in one or more discrete areas on the support surface either alone (i.e., in a discrete area comprising only control probes) or in combination with an analyte probe (i.e., in a discrete area comprising both control and analyte probes).

The sample can comprise a first pool of target nucleic acids labeled with a first enzyme and a second pool of target nucleic acids labeled with a second enzyme. According to this embodiment of the invention, the analyte probes on the support surface can be oligonucleotide or nucleic acid probes. The first and second pools of target nucleic acids can each comprise mRNA transcripts of one or more genes or nucleic acids derived from the mRNA transcripts (e.g., cDNA or cRNA). The concentration of the target nucleic acids in the first and second pools of target nucleic acids can be proportional to the expression level of the genes encoding the target nucleic acid. In this manner, gene expression can be monitored and/or differences in gene expression between two pools of nucleic acids can be determined.

Although nucleic acid probes are described above, the analyte probes can also be polypeptides or any other molecule capable of binding or associating with a target biomolecule in a sample.

The first chemiluminescent substrate and the second chemiluminescent substrate emit chemiluminescent signals which are different and wherein the differences in the emissions are detectable. For example, the emissions from the first and second chemiluminescent substrates can have different emission maxima (i.e., emit different colors).

Detection of the two chemiluminescent signals according to an embodiment of the invention can be accomplished using filters (e.g., optical filters). The first chemiluminescent signal can be detected by filtering the emissions from the support surface with a first filter adapted to reduce the intensity of the second chemiluminescent signal relative to the intensity of the first chemiluminescent signal and detecting the first chemiluminescent signal. The second chemiluminescent signal can be detected by filtering the emissions from the support surface with a second filter adapted to reduce the intensity of the first chemiluminescent signal relative to the intensity of the second chemiluminescent signal and detecting the second chemiluminescent signal.

The composition comprising both the first and second chemiluminescent substrates can be a buffered solution. The buffer can be chosen to optimize detection of the simultaneous emissions from each of the chemiluminescent substrates.

The composition comprising both the first and second chemiluminescent substrates can also comprise a chemiluminescent enhancer. For example, the composition comprising the first and second chemiluminescent substrates can further comprise a chemiluminescent enhancing polymer (e.g., an onium homopolymer or copolymer), one or more enhancing additives (e.g., BSA or β-cyclodextrin), and counterions. The chemiluminescent enhancing polymers, additives and/or counterions can be chosen to optimize detection of simultaneous emissions from chemiluminescent substrates.

The methods described above can be applied to any solid support imaged with chemiluminescence. Exemplary solid supports include, but are not limited to, those disclosed in U.S. patent application Ser. No. 10/046,730, filed Jan. 17, 2002, pending, which application is incorporated herein by reference in its entirety. The solid support can be flexible, semi-rigid, or rigid. Exemplary solid support materials include, but are not limited to, silicon, plastic, glass, membrane coated glass, nylon, nitrocellulose, polyethylsulfone, and pigment impregnated variations thereof. For example, the solid support may comprise an azlactone functional polymer layer. The solid support surface may be two-dimensional (i.e., substantially planar). Alternatively, the support surface may be non-planar. For example, the support surface may comprise undulations resulting from stress relaxation of the solid support to increase feature density as set forth in International Publication No. WO 99/53319, and U.S. patent application Publication Nos. 2001/0053497 A1 and 2001/0053527 A1 which publications are hereby incorporated by reference in their entirety.

The substrate may be porous or non-porous. Exemplary substrates include porous nylon and glass.

As set forth above, the probes on the support may be arranged in an array format wherein a plurality of different probes are disposed in discrete areas on the surface of a solid support. The array can be a microarray having a plurality of probes disposed in a discrete area on the surface of a solid support at a relatively high density. The density of the discrete areas in which probes are disposed on the surface layer, for example, can be at least 50 discrete areas per cm², at least 100 discrete areas per cm², at least 400 discrete areas per cm², at least 1,000 discrete areas per cm², at least 25,000 discrete areas per cm², or at least 50,000 discrete areas per cm².

For purposes of determining surface area, the projected (i.e., 2-dimensional) surface area and not the topographical (i.e., 3-dimensional) surface area of the solid support surface is used. The projected and topographical surface areas can differ significantly for solid support surfaces that are not macroscopically planar. For example, an undulated surface will have a topographical surface area that is greater than its projected (i.e., 2-dimensional) surface area. On the other hand, a macroscopically planar surface will have the same projected and topographical surface areas.

The density of a microarray can also be defined by the center to center distance between adjacent spots on the array which is commonly referred to as the “pitch” or the “probe pitch” of the array. The microarrays according to further embodiments of the invention can have probe pitches of 500 μm or less, 300 μm or less, 250 μm or less, or 80 μm or less. The above ranges are exemplary and other ranges of probe pitch can also be used.

A control probe and/or a control label may be positioned in one or more of the same discrete areas on the support surface along with a probe for a target analyte. The signal from the control label can be used to locate features on the array and/or to normalize the signal from the target analyte. Any of the types of controls disclosed in U.S. patent application Ser. No. 10/050,188, filed Jan. 14, 2002, pending, which is incorporated by reference herein in its entirety, may be used as a control. For example, a control label can be attached to a discrete area on the support surface via attachment of the control label directly to an analyte probe or via attachment to a different molecule attached to the discrete area on the support surface along with the analyte probe. Alternatively, a control label can be attached to a control target capable of binding (e.g., hybridizing) to a control probe attached to one or more discrete areas on the support surface. Any combination or one or more of the above types of controls can be used. For example, a control label and a control probe may both be attached to the support surface and the sample may include a control target (i.e., a target comprising a control label) capable of binding to the control probe.

A composition comprising a first chemiluminescent substrate capable of being cleaved by a first enzyme to produce a first chemiluminescent signal and a second chemiluminescent substrate capable of being cleaved by a second enzyme to produce a second chemiluminescent signal is also provided according to a further embodiment of the invention. According to this embodiment of the invention, the first and second chemiluminescent signals are different (e.g. have different emission maxima). The composition can be a buffered solution wherein the buffer is adapted to maximize simultaneous emissions from each of the chemiluminescent substrates. The composition can also comprise a chemiluminescent quantum yield enhancing agent, additives and/or counterions chosen to maximize simultaneous emissions.

Any chemiluminescent, enzyme-activatable compound can be used as a chemiluminescent substrate. For example, the chemiluminescent substrate can be a luminol, an acridan ester or thioester, an enol phosphate such as an acridan enol phosphate, or a 1,2-dioxetane compound. The 1,2-dioxetane compound can be induced to decompose to yield a moiety in an excited state having a heteropolar character that makes it susceptible to environmental effects, particularly to dampening or diminution of luminescence in a polar protic environment. The chemiluminescent compound can be used to determine the presence, concentration or structure of a substance in a polar protic environment, particularly a substance in an aqueous sample.

Among the most effective compounds for this purpose are the stabilized, enzyme-cleavable 1,2-dioxetanes. A number of classes of these chemiluminescent enzyme-triggerable 1,2-dioxetanes, containing a variety of stabilizing functions are known. For example, spiro-bound polycycloalkyl groups either unsubstituted, substituted, or containing sp2 centers are taught in U.S. Pat. Nos. 5,112,960, 5,225,584, and 6,461,876, which are hereby incorporated by reference in their entirety. In addition, branched dialkyl-stabilized, enzyme-triggerable dioxetanes are taught in U.S. Pat. No. 6,284,899, which is also incorporated by reference in its entirety. Substituted furan and pyran-stabilized enzyme-triggerable dioxetanes are taught in U.S. Pat. No. 5,731,445, and European Patent Application Nos. EP 0943618 and EP 1038876, which are also incorporated by reference herein in their entirety. Any of the chemiluminescent substrates disclosed in the aforementioned publications can be used.

A dioxetane having a stabilizing moiety can be used as a chemiluminescent substrate. The stabilizing moiety can be chosen based on the requirements of the application. Further, the dioxetanes may also be further substituted with one or more electron withdrawing (e.g. chlorine or fluorine), electron donating (e.g. alkyl or methoxy) groups, or deuterium atoms at any position. This allows tailoring of the quantum yield, emission half-life or pKa [Star dioxetanes] of the enzyme product. The dioxetane can be protected with an enzyme-labile group to form an enzyme cleavable substrate.

As set forth above, stabilized 1,2-dioxetanes (e.g., 1,2-dioxetanes stabilized with an adamantyl group) can be used as the chemiluminescent substrate. This class of dioxetanes can be represented by the following general formula: 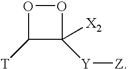
In the above formula, T represents an unsubstituted or substituted cycloalkyl, aryl, polyaryl or heteroatom group (e.g., an unsubstituted cycloalkyl group having from 6 to 12 ring carbon atoms, inclusive); a substituted cycloalkyl group having from 6 to 12 ring carbon atoms, inclusive, and having one or more substituents which can be an alkyl group having from 1 to 7 carbon atoms, inclusive, or a heteroatom group which can be an alkoxy group having from 1 to 12 carbon atoms, inclusive, such as methoxy or ethoxy, a substituted or unsubstituted aryloxy group, such as phenoxy or carboxyphenoxy, or an alkoxyalkyloxy group, such as methoxyethoxy or polyethyleneoxy, or a cycloalkylidene group bonded to the 3-carbon atom of the dioxetane ring through a spiro linkage and having from 6 to 12 carbon atoms, inclusive, or a fused polycycloalkylidene group bonded to the 3-carbon of the dioxetane ring through a spiro linkage and having two or more fused rings, each having from 5 to 12 carbon atoms, inclusive, e.g., an adamant-2-ylidene group.

The symbol Y represents a chromophoric group capable of producing a luminescent substance, which can emit light from an excited energy state upon dioxetane decomposition initiated by enzyme activation.

The symbol X₂ represents hydrogen or an alkyl, aryl, aralkyl, alkaryl, heteroalkyl, heteroaryl, cycloalkyl or cycloheteroalkyl group, e.g., a straight or branched chain alkyl group having from 1 to 7 carbon atoms, inclusive; a straight or branched chain hydroxyalkyl group having from 1 to 7 carbon atoms, inclusive, or an —OR group in which R is a C₁-C₂₀ unbranched or branched, unsubstituted or substituted, saturated or unsaturated alkyl, cycloalkyl, cycloalkenyl, aryl, aralkyl or aralkenyl group, fused ring cycloalkyl, cycloalkenyl, aryl, aralkyl or aralkenyl group, or an N, O or S hetero atom-containing group, or an enzyme-cleavable group containing a bond cleavable by an enzyme to yield an electron-rich moiety bonded to the dioxetane ring. According to one embodiment of the invention, X₂ can be a methoxy group or a trifluoroethoxy group (—OCH₂CF₃).

The symbol Z in the above formula represents an enzyme-cleavable group containing a bond cleavable by an enzyme to yield an electron-rich moiety bonded to the dioxetane ring, e.g., a bond which, when cleaved, yields an oxygen anion, a sulfur anion, a nitrogen anion, or an amido anion such as a sulfonamido anion.

An exemplary chemiluminescent substrate is the CDP-Star® substrate (Applied Biosystems, Foster City, Calif.) which is represented by the following chemical formula:

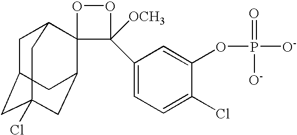

A further exemplary chemiluminescent substrate is the TFE-CDP-Star® substrate (Applied Biosystems, Foster City, Calif.) which is represented by the following chemical formula:

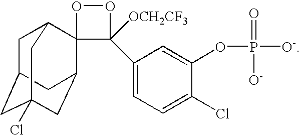

A further exemplary chemiluminescent substrate is Galacton-Star® substrate. Galacton-Star® is a registered trademark of Applied Biosystems, Foster City, Calif.

Deuterated dioxetanes can also be used as chemiluminescent substrates. Deuteration of the chemiluminescent dioxetane substrate can result in an increased chemiluminescent signal.

Chemiluminescent substrates other than dioxetanes can also be used. Exemplary chemiluminescent substrates include, but are not limited to, acridan ester or thioester substrates, enol phosphate substrates such as acridan enol phosphates, and luminol substrates. When an acridan ester or thioester substrate or a luminol substrate is employed, the target molecules can be labeled with an oxidative enzyme such as a peroxidase (e.g., horseradish peroxidase), a catalase or a xanthine oxidase. Enol phosphate substrates such as acridan enol phosphates for alkaline phosphatase can also be used.

The first and second chemiluminescent substrates can both be 1,2-dioxetanes that emit detectably different chemiluminescent signals. Alternatively, the first chemiluminescent substrate can be a 1,2-dioxetane chemiluminescent substrate and the second chemiluminescent substrate can be a non-dioxetane chemiluminescent substrate (e.g., an acridan or luminol substrate). According to this embodiment, each of the substrates can have a different enzyme-cleavable group (i.e., a group cleavable by a different enzyme).

Any type of probe that is capable of recognizing and binding to a target molecule in the sample can be used. Exemplary probes for nucleic acid targets include, but are not limited to, oligonucleotide probes and cDNA probes. For nucleic acid hybridization assays, the probe comprises a material that is capable of hybridizing with the target nucleic acid. Exemplary probes for protein or polypeptide targets include, but are not limited to, polypeptide probes, aptamer probes, and antibody probes.

The targets in the sample can be labeled with an enzyme capable of cleaving an enzyme labile group on a chemiluminescent substrate. Alternatively, the target can be labeled with a moiety capable of binding with an enzyme conjugate comprising an enzyme capable of cleaving an enzyme labile group on a chemiluminescent substrate. When the target is assayed indirectly, the target molecules can be labeled with a ligand and an enzyme conjugate capable of binding the ligand can be employed. Exemplary ligand/enzyme conjugate pairs which can be used include, but are not limited to, digoxigenin/antidigoxigenin:enzyme conjugates, biotin/streptavidin:enzyme conjugates, streptavidin/biotin:enzyme conjugates; and fluorescein/antifluorescein:enzyme conjugates.

Alternatively, the target can be unlabeled and detected by hybridization with a second labeled probe that binds to a portion of the target molecule different from that bound by the capture probe on the support surface. The second labeled probe can be labeled directly with an enzyme or with various ligands as set forth above and detected with an enzyme conjugate capable of binding the ligand.

Although the specific embodiments described above involve the simultaneous generation of two chemiluminescent signals, additional chemiluminescent signals can also be used. Therefore, according to a further embodiment, three or more chemiluminescent signals can be simultaneously generated.

The foregoing description is by way of example only and is not intended to be limiting. Although specific embodiments have been described herein for purposes of illustration, various modifications to these embodiments can be made without the exercise of inventive faculty. All such modifications are within the spirit and scope of the appended claims.

1. A method of detecting chemiluminescent emissions on a solid support, the method comprising:
contacting a surface layer of the solid support with a substrate composition comprising a first chemiluminescent substrate capable of being activated by a first enzyme to produce a first chemiluminescent signal and a second chemiluminescent substrate capable of being activated by a second enzyme to produce a second chemiluminescent signal; and
detecting first and second chemiluminescent signals on the surface layer of the solid support;
wherein a plurality of probes are disposed in a plurality of discrete areas on the surface layer at a density of at least 50 discrete areas per cm², wherein at least some of the probes are bound to a first enzyme conjugate comprising the first enzyme, and wherein at least some of the probes are bound to a second enzyme conjugate comprising the second enzyme.

2. The method of claim 1, wherein the composition comprising the first and second chemiluminescent substrates is contacted with the surface layer in the presence of a composition comprising a chemiluminescent quantum yield enhancing material.

3. The method of claim 1, wherein the discrete areas comprise one or more control probes and wherein the first enzyme conjugate is bound to a control probe.

4. The method of claim 3, further comprising quantifying the amount of the second chemiluminescent signal.

5. The method of claim 4, wherein quantifying comprises comparing the intensity of the first chemiluminescent signal to the intensity of the second chemiluminescent signal.

6. The method of claim 3, wherein a plurality of different probes are 5 disposed on the support surface in different discrete areas and wherein detecting comprises detecting the location on the support surface of first and second chemiluminescent signals.

7. The method of claim 6, wherein control probes are located in one or more discrete areas on the support surface.

8. The method of claim 6, wherein control probes are co-located in one or more of the same discrete areas as probes for a target molecule.

9. The method of claim 1, wherein detecting comprises detecting the location on the support surface of first and second chemiluminescent signals.

10. The method of claim 9, wherein the plurality of discrete areas comprise oligonucleotide or nucleic acid probes.

11. The method of claim 1, further comprising:
contacting the support surface with a sample comprising first target molecules labeled with a first label and second target molecules labeled with a second label prior to contacting the support surface with the substrate composition.

12. The method of claim 11, wherein the first target molecules are labeled with the first enzyme to form the first enzyme conjugate and the second target molecules are labeled with the second enzyme to form the second enzyme conjugate.

13. The method of claim 1 1, wherein the first target molecules are labeled with a moiety capable of binding to the first enzyme conjugate and the second target molecules are labeled with a moiety capable of binding to the second enzyme conjugate.

14. The method of claim 11, wherein the first target molecules comprise a first pool of target nucleic acids and wherein the second target molecules comprise a second pool of target nucleic acids.

15. The method of claim 14, wherein the first and second pools of target nucleic acids each comprise mRNA transcripts of one or more genes or nucleic acids derived from mRNA transcripts of one or more genes.

16. The method of claim 14, wherein the first and second pools of target nucleic acids each comprise cDNA or cRNA derived from mRNA transcripts.

17. The method of claim 14, wherein the concentration of the target nucleic acids in the first and second pools of target nucleic acids is proportional to the expression level of the genes encoding the target nucleic acid.

18. The method of claim 1 1, wherein the probes comprise a control probe and wherein the first enzyme conjugate is bound to the control probe.

19. The method of claim 18, wherein the plurality of different probes comprise oligonucleotide or nucleic acid probes and wherein the sample comprises a pool of target nucleic acids labeled with the second enzyme.

20. The method of claim 19, wherein the pool of target nucleic acids comprises mRNA transcripts of one or more genes or nucleic acids derived from the mRNA transcripts of the one or more genes.

21. The method of claim 20, wherein the pool of target nucleic acids comprises cDNA or cRNA derived from mRNA transcripts of the one or more genes.

22. The method of claim 21, wherein the concentration of each of the target nucleic acids in the pool of target nucleic acids is proportional to the expression level of each of the genes encoding the target nucleic acid.

23. The method of claim 1, wherein the density of discrete areas on the surface layer is at least 100 discrete areas per cm².

24. The method of claim 1, wherein the density of discrete areas on the surface layer is at least 1,000 discrete areas per cm².

25. The method of claim 1, wherein the density of discrete areas on the surface layer is at least 25,000 discrete areas per cm².

26. The method of claim 1, wherein the density of discrete areas on the surface layer is at least 50,000 discrete areas per cm².

27. The method of claim 1, wherein the support surface further comprises a fluorescent control.

28. The method of claim 1, wherein the first chemiluminescent signal and the second chemiluminescent signal have different emission maxima.

29. The method of claim 28, wherein detecting first and second chemiluminescent signals comprises:
filtering the emissions from the support surface with a first filter adapted to reduce the intensity of the second chemiluminescent signal relative to the intensity of the first chemiluminescent signal;
detecting the first chemiluminescent signal;
filtering the combined signal from the support surface with a second filter adapted to reduce the intensity of the first chemiluminescent signal relative to the intensity of the second chemiluminescent signal; and
detecting the second chemiluminescent signal.

30. The method of claim 1, wherein the composition comprising the first and second chemiluminescent substrates is a buffered solution.

31. The method of claim 1, further comprising washing the surface layer of the solid support before contacting the surface layer with the substrate composition.

32. The composition of claim 1, wherein the first and second chemiluminescent substrates are both 1,2-dioxetanes.

33. The composition of claim 1, wherein the first chemiluminescent substrate is a 1,2-dioxetane substrate and the second chemiluminescent substrate is selected from the group consisting of an acridan ester substrate, an acridan thioester substrate, an enol phosphate substrate, an acridan enol phosphate substrate, and a luminol substrate.

34. A composition comprising a first chemiluminescent substrate capable of being activated by a first enzyme to produce a first chemiluminescent signal and a second chemiluminescent substrate capable of being activated by a second enzyme to produce a second chemiluminescent signal, wherein the first and second chemiluminescent signals are different.

35. The composition of claim 34, wherein the composition is a buffered solution.

36. The composition of claim 34, further comprising a chemiluminescent quantum yield enhancing agent, additive and/or counterion.

37. The composition of claim 34, wherein the first and second chemiluminescent substrates are each 1,2-dioxetanes.

38. The composition of claim 34, wherein the first chemiluminescent substrate is a 1,2-dioxetane substrate and the second chemiluminescent substrate is selected from the group consisting of an acridan ester substrate, an acridan thioester substrate, an enol phosphate substrate, an acridan enol phosphate substrate, and a luminol substrate.

39. The method of claim 2, wherein the composition comprising the chemiluminescent quantum yield enhancing material further comprises an additive selected from the group consisting of BSA, cyclodextrins, negatively charged salts, alcohols, polyols, poly(2-ethyl-Z-oxazoline), zwitterionic surfactants, anionic surfactants, cationic surfactants, and neutral surfactants.

40. The method of claim 2, wherein the composition comprising the chemiluminescent quantum yield enhancing material further comprises one or more counterion moieties selected from the group consisting of halide, sulfate, alkylsulfonate, triflate, arylsulfonate, perchlorate, alkanoate, arylcarboxylate and combinations thereof.

41. The method of claim 13, wherein the first or second enzyme conjugate is an antidigoxigenin:enzyme conjugate and wherein the corresponding target molecules are labeled with digoxigenin.

42. The method of claim 14, wherein the first or second pools of target nucleic acids are labeled with digoxigenin and the corresponding enzyme conjugate is an antidigoxigenin:enzyme conjugate.

43. The method of claim 42, wherein the pool of target nucleic acids labeled with digoxigenin comprises cDNA.

44. The method of claim 2, wherein the chemiluminescent quantum yield enhancing material is an onium polymer selected from the group consisting of poly(vinylbenzylammonium salts), poly(vinylbenzylphosphonium salts) and poly(vinylbenzylsulfonium salts).

45. The method of claim 2, wherein the chemiluminescent quantum yield enhancing material is an onium copolymer.
